(19) United States
(12) Patent Application Publication
(10) Pub. No.: US 2010/0137623 A1
(43) Pub. Date: Jun. 3, 2010

(54) STABLE CATALYST PRECURSOR OF RH COMPLEX CATALYSTS

(75) Inventors: Detlef Selent, Rostock (DE); Armin Boerner, Rostock (DE); Klaus-Diether Wiese, Haltern am see (DE); Dieter Hess, Marl (DE); Dirk Fridag, Haltern am see (DE)

Correspondence Address: OBLON, SPIVAK, MCCLELLAND MAIER & NEUSTADT, L.L.P., 1940 DUKE STREET, ALEXANDRIA, VA, 22314, US

(73) Assignee: EVONIK OXENO GMBH, Marl (DE)

(21) Appl. No.: 12/594,602

(22) PCT Filed: Mar. 19, 2008
(86) PCT No.: PCT/EP2008/053254

§ 371 Date: Oct. 5, 2009

(30) Foreign Application Priority Data
May 18, 2007 (DE) 102007023514.5

(51) Int. Cl. C07C 45/50; C07F 15/00; B01J 31/12

(52) U.S. Cl. 556/13; 568/454; 502/155

(57) ABSTRACT

The present invention relates to the preparation and use of stable catalyst precursors of rhodium complex catalysts.

The present invention relates to the preparation and use of catalyst precursors of rhodium complex catalysts.

The use of homogeneous rhodium complex catalysts in various processes of industrial organic chemistry has been known for a long time. In particular, the use of rhodium-phosphine, -phosphite, -phosphonite or -phosphinite ligand complexes in processes for the hydroformylation of olefins has been adequately described. However, processes using catalysts comprising rhodium and ligands having at least one phosphorus-oxygen bond have hitherto not become widespread. This is presumably due to the fact that these catalysts are quite expensive and have only a low stability to relatively high temperatures, oxygen and/or water.

In industrial processes, the active catalyst is, for cost reasons and/or because of the difficulty of handling it, not introduced into the process in pure form but is instead generated in the hydroformylation reactor from one or more suitable precursor(s) under the reaction conditions of the hydroformylation.

The suitability of a potential catalyst precursor depends on various factors. These factors include: commercial availability and price, storage stability, suitable handability in respect of transport and introduction into the reactor, compatibility with cocatalysts, solubility in the desired reaction medium, rapid catalyst activation or rapid start of the reaction with a minimal induction period and the absence of negative effects of the by-products formed during catalyst formation on the production plant or the productivity of the reaction.

It was therefore an object of the invention to discover a catalyst precursor which can be handled readily in respect of transport and introduction into the reactor, can easily be converted into the active catalyst and does not form any substances which reduce the catalyst stability and/or the reactivity and/or the selectivity.

It is known that metal halides used as precursors can liberate hydrohalic acids which have a corrosive effect on plant components. Precursors which are salts of organic Brönsted acids, e.g. acetylacetonates, carboxylates, alkoxides and hydroxides (amides), likewise liberate the corresponding proton-active compound, i.e. acetylacetone, carboxylic acid, alcohol or water (amines), during the course of catalyst activation. This can be brought about by various processes, but in the case of transition metal compounds is preferably effected by reduction with hydrogen.

The liberation of proton-active compounds does not have to be disadvantageous. However, in the case of particular reactions it is not desired. This applies, for example, to reactions in which a required cocatalyst or the catalyst is destroyed or else altered in an undesirable way by the proton-active compounds in the case of relatively long reaction times. This applies, for example, to the rhodium-catalyzed hydroformylation of olefins in the presence of modifying phosphite compounds as cocatalysts. Phosphites generally tend to undergo reactions with proton-active compounds. In the context of the hydroformylation reaction, the compounds used as cocatalysts are also referred to as ligands.

One possibility would be to remove the undesirable activation products by distillation. However, this can generally not be carried out in practice.

In addition, precursors which are salts of organic Brönsted acids are often present in the wrong oxidation state of the rhodium, so that the rhodium firstly has to be reduced in the preformation. The reduction of the rhodium can form compounds which are damaging to the cocatalyst. Accordingly, for the hydroformylation reaction or other reactions carried out in the presence of trivalent phosphorus compounds having at least one oxygen-phosphorus bond, for example phosphites, it would be desirable to have catalyst precursors which do not contain any anions which can form generally damaging protic acids in the reaction with hydrogen or in catalyst activation.

Furthermore, precursors which contain ligands which can be removed only with difficulty because of a high complex formation constant, e.g. HRh(TPP)₃(CO), are also unfavourable for the activity and/or regioselectivity of the catalyst.

Products of an ortho metallation are known for various transition metal complexes of phosphines and also phosphites. EP 1249441 A1 has described a rhodium complex formed from binol-diphosphite as intermediate and catalyst depot in a continuous hydroformylation process. Here, the ortho-metallated complex is formed from the catalytically active hydrido complex by H abstraction by the olefin.

It has surprisingly been found that the precursors for rhodium complex catalysts are very stable and thus able to be handled readily when they have the structure I. These compounds are very useful catalyst precursors since they do not form any protic acids or other undesirable by-products during catalyst activation and have good solubilities and the ligands in compounds of the structure I can easily be displaced by ligands of the desired catalyst system.

The present invention accordingly provides a catalyst precursor comprising a rhodium complex of the formula I

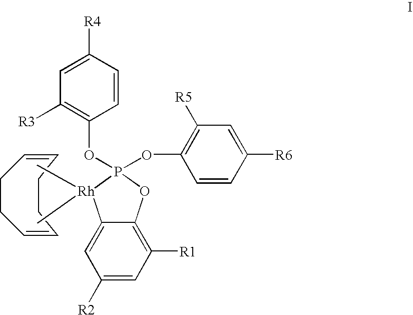

where R1 to R6=H or a C₁-C₄-alkyl or C₁-C₄-alkoxy radical or
R1, R2, R4 and R6=H or a C₁-C₄-alkyl or C₁-C₄-alkoxy radical and
R3=R5=O, NR where R═H or C₁-C₄-alkyl, or (CH₂)ₙ where n=0-2.

The present invention likewise provides a mixture containing a catalyst precursor according to the invention and at least one organophosphorus ligand.

In addition, the present invention provides for the use of a catalyst precursor according to the invention for preparing a catalyst for hydrocyanation, hydroacylation, hydroamidation, hydrocarboxyalkylation, aminolysis, alcoholysis, carbonylation, isomerization or a hydrogen transfer process and also a process for the hydroformylation of olefins, which is characterized in that a catalyst obtained from a catalyst precursor according to the invention is used.

The catalyst precursors of the invention have the advantage that they have a high storage stability. In particular, the catalyst precursors have a relatively high stability to thermal stress, oxidation or hydrolysis. The good storage stability makes the catalyst precursors of the invention very suitable for keeping in readiness as catalyst precursors for processes in which metal-organophosphorus ligand complexes are to be used or have to be used. The corresponding metal-organophosphorus ligand complex catalysts can be produced very simply from the catalyst precursors of the invention by addition of desired ligands and synthesis gas. A particular advantage results from the fact that no damaging protic acids are formed from the catalyst precursors of the structure I during catalyst formation under synthesis gas, but instead the rhodium-carbon bond present is cleaved by hydrogen and converted into an Rh—H bond and a C—H bond which can be considered to be inert under the conditions of the abovementioned reactions. This catalyst complex formation proceeds quickly even at room temperature when using precursors of the structure I. This results in the advantage, for instance for a continuous process with further introduction of catalyst, that the time required for formation of the catalyst from the precursor becomes very short compared to the catalyst residence time and makes better control of the process possible.

The catalyst precursors of the invention are characterized in that they represent a rhodium complex of the formula I

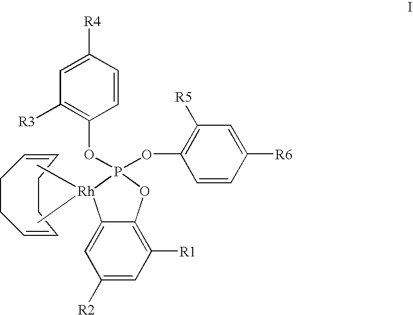

where R1 to R6=H or a C₁-C₄-alkyl or C₁-C₄-alkoxy radical or
R1, R2, R4 and R6=H or a C₁-C₄-alkyl or C₁-C₄-alkoxy radical and
R3=R5=O, NR where R═H or C₁-C₄-alkyl, or (CH₂)ₙ where n=0-2.

Preference is given to all of the radicals R1 to R6 being a C₁-C₄-alkyl radical. It can be advantageous for at least one of the radicals R1 to R6 to be a tert-butyl radical.

Very particular preference is given to all radicals R1 to R6 being a tert-butyl radical. Very particular preference is given to the catalyst precursor of the invention being a complex of the formula II

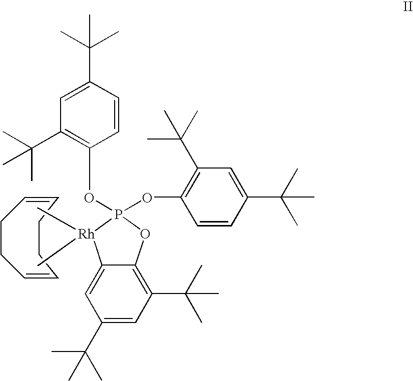

The preparation of the catalyst precursor of the general formula I is carried out by means of the reaction known per se of a cyclooctadiene-rhodium complex, preferably allyl(1,5-cyclooctadiene)rhodium, with a phosphite of the general formula III.

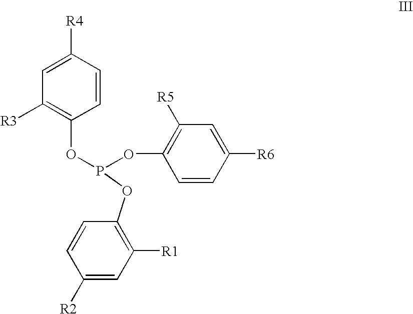

The catalyst precursors of the invention can be used as pure substances or as a mixture. The mixtures according to the invention containing the catalyst precursors of the invention can comprise the catalyst precursor together with, in particular, one or more solvents. Such solvents can be solvents which are inert in respect of the reaction for which the catalyst precursor is to be used after conversion into the catalyst. If one of the starting materials is used as solvent in the reactions, it can be advantageous also to provide one of these starting materials used as solvent in the is reaction as solvent in the mixture according to the invention. If the catalyst precursor is used, for example, for forming the catalyst for a hydroformylation reaction, it can be advantageous to use the olefin used in the hydroformylation, e.g. a C₄-, C₅-, C₆-, C₇-, C₈-, C₉-, C₁₀-, C₁₁-, C₁₂-, C₁₃-, C₁₄-, C₁₅-, C₁₆- or C₂₀-olefin, as solvent. If the mixture of the invention is to comprise an inert solvent, it is possible to use, for example, toluene in the case of hydroformylation.

Apart from the catalyst precursors and, if desired, a solvent, the mixtures of the invention can comprise further ligands, in particular organophosphorus ligands.

The catalyst precursor of the invention can be used as precursor for preparing a catalyst for hydrocyanation, hydroacylation, hydroamidation, hydrocarboxyalkylation, aminolysis, alcoholysis, carbonylation, isomerization or a hydrogen transfer process. To prepare the actual catalyst, it has been found to be advantageous to react the catalyst precursor with the ligand provided for the metal complex catalyst under reaction conditions in the presence of the ligand, resulting in complete or at least partial ligand exchange taking place.

A process according to the invention for the hydroformylation of olefins in which a catalyst obtained from a catalyst precursor according to the invention is used is described by way of example below.

In the process of the invention for the hydroformylation of olefins, preference is given to using olefins having from 2 to 25 carbon atoms, particularly preferably from 6 to 12 carbon atoms and very particularly preferably 8, 9, 10, 11 or 12 carbon atoms.

In addition to the complex catalysts, free organophosphorus ligands can be present in the reaction mixture of the hydroformylation if desired. The free ligands and the ligands bound in the complex catalysts are preferably selected from among phosphines, phosphites, phosphinites, phosphonites. The ligands can comprise one or more phosphine, phosphite, phosphonite and phosphinite groups. It is likewise possible for the ligands to comprise two or more different groups selected from among phosphine, phosphite, phosphonite or phosphinite groups. In particular, the ligands can be bisphosphites, bisphosphines, bisphosphonites, bisphosphinites, phosphine-phosphites, phosphine-phosphonites, phosphine-phosphinites, phosphite-phosphonites, phosphite-phosphinites or phosphonite-phosphinites. The ligands of the complex catalyst and the free ligands can be identical or different. Preference is given to the organophosphorus ligands of the complex catalyst and the free ligands being identical. Examples of complex catalysts or ligands which can be used and their preparation and use in hydroformylation may be found in, for example, EP 0 213 639, EP 0 214 622, EP 0 155 508, EP 0 781 166, EP 1209164, EP 1201675, DE 10114868, DE 10140083, DE 10140086, DE 10210918.

Examples of preferred ligands are:

Phosphines: triphenylphosphine, tris(p-tolyl)phosphine, tris(m-tolyl)phosphine, tris(o-tolyl)phosphine, tris(p-methoxyphenyl)phosphine, tris(p-dimethylaminophenyl)phosphine, tris(cyclohexyl)phosphine, tris(cyclopentyl)phosphine, triethylphosphine, tris(1-naphthyl)phosphine, tribenzylphosphine, tri-n-butylphosphine, tri-t-butylphosphine.

Phosphites: trimethyl phosphite, triethyl phosphite, tri-n-propyl phosphite, tri-1-propyl phosphite, tri-n-butyl phosphite, tri-1-butyl phosphite, tri-t-butyl phosphite, tris(2-ethylhexyl) phosphite, triphenyl phosphite, tris(2,4-di-t-butylphenyl) phosphite, tris(2-t-butyl-4-methoxyphenyl) phosphite, tris(2-t-butyl-4-methylphenyl) phosphite, tris(p-cresyl) phosphite.

Phosphonites: methyldiethoxyphosphine, phenyldimethoxyphosphine, phenyldiphenoxyphosphine, 2-phenoxy-2H-dibenz[c,e][1,2]oxaphosphorine and its derivatives in which the hydrogen atoms have been completely or partly replaced by alkyl and/or aryl radicals or halogen atoms.

Customary phosphinite ligands are diphenyl(phenoxy)phosphine and its derivatives, diphenyl(methoxy)phosphine and diphenyl(ethoxy)phosphine.

Particular preference is given to using complex catalysts containing a mixed anhydride of phosphorous acid and an arylcarboxylic or hydroxyarylcarboxylic acid in the process of the invention for hydroformylation. Acylphosphites or ligands comprising acylphosphite groups, their preparation and their use in hydroformylation are described, for example, in DE 100 53 272, which is hereby incorporated by reference into the disclosure of the present invention. Heteroacylphosphites and ligands comprising heteroacylphosphite groups, their preparation and their use in hydroformylation are described, for example, in DE 10 2004 013 514.

Among the acylphosphites described in DE 100 53 272, the acylphosphites shown below are particularly preferred organophosphorus ligands which can be present as catalyst complex ligand and/or as free ligand in a hydroformylation process according to the invention.

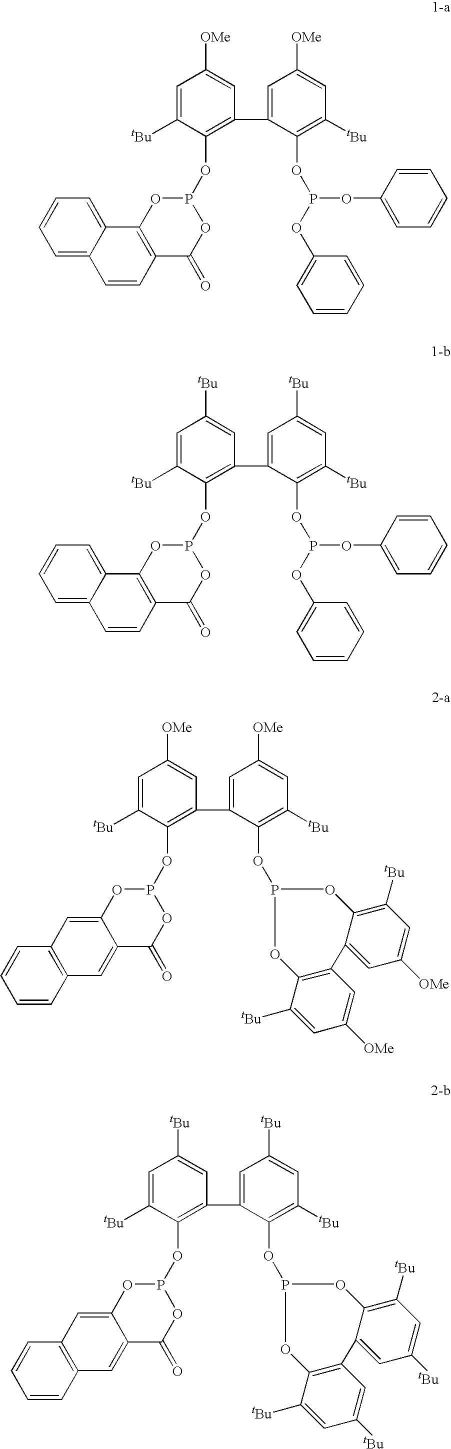

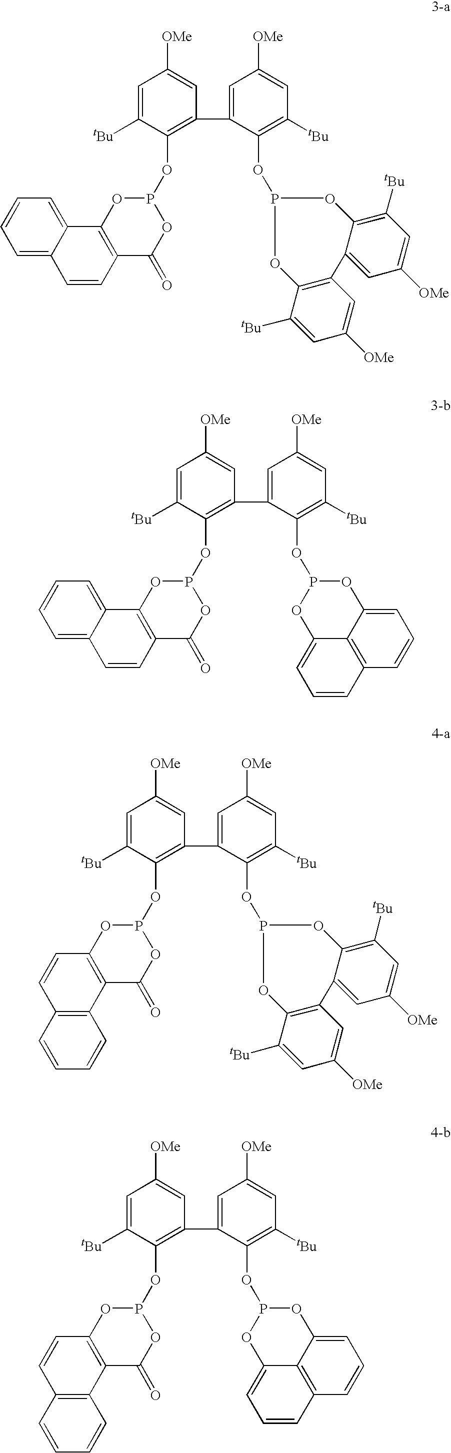

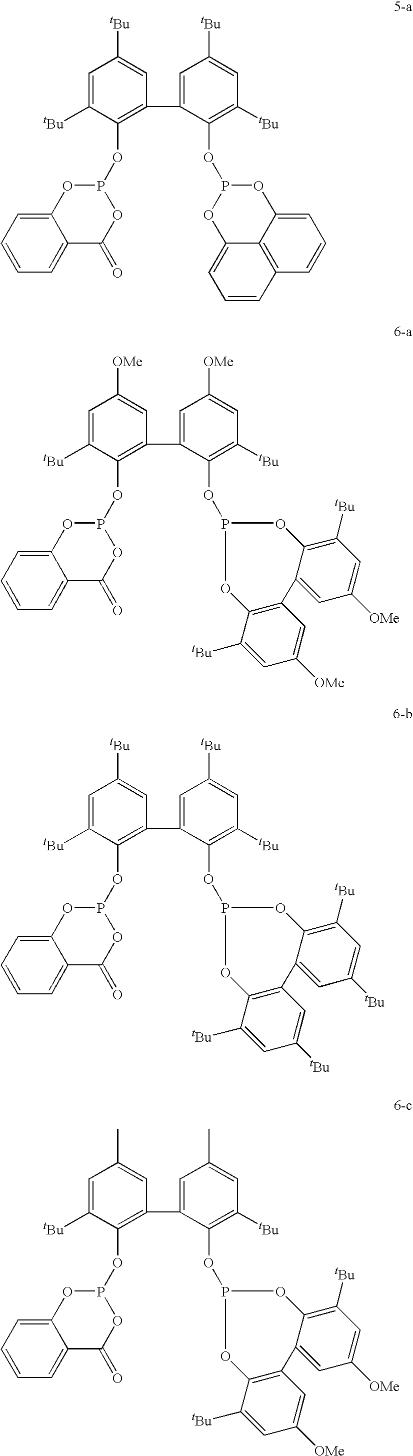

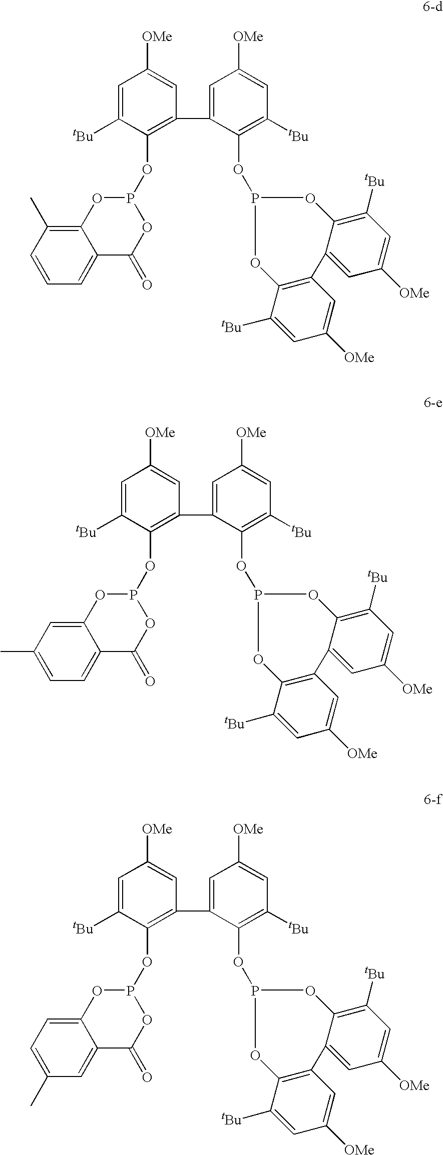

In a further preferred embodiment of the process of the invention, the heteroacylphosphites of the general formula (I) described in DE 10 2004 013 514 can be used as ligands.

The hydroformylation process of the invention is preferably carried out using from 1 to 500 mol, preferably from 1 to 200 mol and particularly preferably from 2 to 50 mol, of organophosphorus ligand per mole of rhodium. Fresh organophosphorus ligands can be added at any point in time during the hydroformylation reaction in order to to keep the concentration of free heteroacylphosphite, i.e. heteroacylphosphite which is not coordinated to the metal, constant.

The concentration of the metal in the hydroformylation mixture is preferably in the range from 1 ppm by mass to 1000 ppm by mass, more preferably in the range from 5 ppm by mass to 300 ppm by mass, based on the total mass of the hydroformylation mixture.

The hydroformylation reactions carried out using the organophosphorus ligands or the corresponding metal complexes can be carried out by known methods, as described, for example, in J. FALBE, “New Syntheses with Carbon Monoxide”, Springer Verlag, Berlin, Heidelberg, N.Y., page 95 ff., (1980). The olefin compound(s) is(are) reacted with a mixture of CO and H₂ (synthesis gas) in the presence of the catalyst to form the aldehydes having one more carbon atom.

The reaction temperatures are preferably from 40° C. to 180° C. and more preferably from 75° C. to 140° C. The pressures under which the hydroformylation proceeds are preferably from 0.1 to 30 MPa of synthesis gas and more preferably from 1 to 6.4 MPa. The molar ratio of hydrogen to carbon monoxide (H₂/CO) in the synthesis gas is preferably from 10/1 to 1/10 and more preferably from 1/1 to 2/1.

The catalyst or the ligand is preferably present in homogeneously dissolved form in the hydroformylation mixture comprising starting materials (olefins and synthesis gas) and products (aldehydes, alcohols, high boilers formed in the process). A solvent, which may also be selected from among the starting materials (olefins) and products (aldehydes) of the reaction, may be additionally present. Further possible solvents are organic compounds which do not interfere in the hydroformylation reaction and can preferably be separated off again easily, e.g. by distillation or extraction. Such solvents can be, for example, hydrocarbons such as toluene.

The starting materials for the hydroformylation are olefins or mixtures of olefins having from 2 to 25 carbon atoms and a terminal or internal C═C double bond. Preferred starting materials are, in particular, α-olefins such as propene, 1-butene, 2-butene, 1-hexene, 1-octene and oligomers of butene (isomer mixtures), in particular di-n-butene and tri-n-butene.

The hydroformylation can be carried out continuously or batchwise. Examples of industrial embodiments are stirred vessels, bubble columns, jet nozzle reactors, tube reactors or loop reactors, some of which can be cascaded and/or provided with internals. The reaction can be carried out in a single pass or in a plurality of stages.

The work-up of the hydroformylation mixture can be carried out in various ways known from the prior art. The work-up is preferably carried out by firstly separating off all gaseous constituents from the hydroformylation mixture. This is usually followed by the hydroformylation products and any unreacted starting olefins being separated off. This separation can be achieved, for example, by use of flash evaporators or falling film evaporators or distillation columns. As residue, it is possible to obtain a fraction which comprises essentially the catalyst and any high boilers formed as by-products. This fraction can be recirculated to the hydroformylation.

The following examples illustrate the invention without restricting the scope of protection which is defined by the claims and the description.

### EXAMPLE 1

### Preparation of II

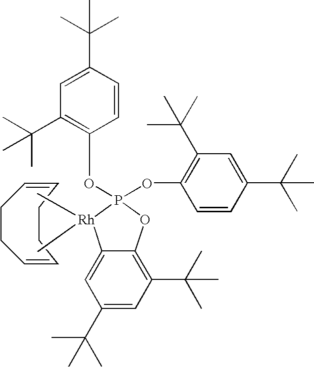

A solution of tris(2,4-di-tert-butylphenyl) phosphite (1.398 g; 2.16 mmol) in toluene (15 ml) is added dropwise to a solution of allyl(1,5-cyclooctadiene)rhodium (0.545 g; 2.16 mmol) in pentane (15 ml) at room temperature while stirring. After the addition of phosphite is complete, the reaction solution is stirred for 3 hours and evaporated to dryness under reduced pressure. The residue obtained is dried under reduced pressure for 2 hours and subsequently dissolved in warm toluene (12 ml). Storage of the solution for 1 day at room temperature, subsequent crystallization over a period of 3 days by storage in a refrigerator, filtration, washing of the residue with cooled hexane (6 ml) and drying at 60° C. for 3 hours give a spectroscopically pure product as monotoluene adduct in the form of bright orange-red crystals.

Yield: 1.36 g (1.433 mmol; 65% of theory).

Elemental analysis (calculated for C₅₇H₈₂O₃PRh=949.155 g/mol) C, 71.41 (72.13); H, 9.56 (8.71); P, 3.28 (3.26); Rh, 10.88 (10.84) %.

³¹P-NMR (CD₂Cl₂): δ 147.0 (d, ¹J_{P—Rh}=317.7 Hz) ppm. ¹H-NMR (CD₂Cl₂): δ 1.18 (s, 9H); 1.37 (s, 18H); 1.42 (s, 9H); 1.57 (s, 18H); 2.04 (m, 2H); 2.29 (m, 6H); 2.41 (s, 3H); 4.31 (s, 2H); 6.01 (s, 2H); 7.09-7.83 (m, 13H) ppm.

Evaluation of an X-ray crystal structure analysis confirms the course of the reaction to as formulated below:

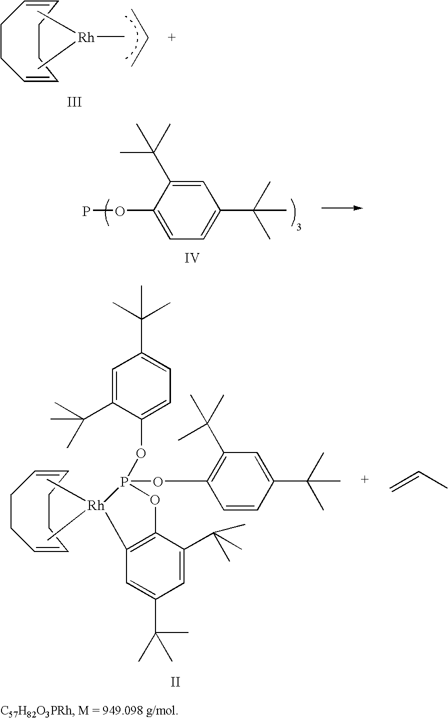

### EXAMPLE 2

The complex II is a very good precursor for hydroformylation. When two equivalents of ligand 6-a are used, complete conversion and a selectivity of 62.8% are obtained for the n-octenes at 120° C., 20 bar, 100 ppm of Rh, toluene. The gas consumption curve is analogous to those for the batches using [acacRh(COD)] as precursor, which indicates rapid formation of a rhodium hydride with hydrogenolysis of the Rh-aryl bond.

Experimental Conditions: 120° C., 20 bar, 8 h, 100 ppm of Rh, 10.7 g of dibutene and 35.2 g of toluene.

### Precursors:

[acacRh(COD)] and Precursor II

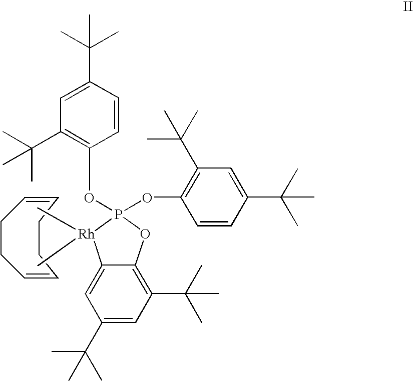

The Rh/ligand ratio was set assuming complete hydrogenolysis of the metallated to precursor II, as follows:

Rh/II=1:1

| | | Conversion | |
|---|---|---|---|
| No. | Precursor | (%) | n-% |
| 1 | [acacRh(COD)] | 53.5 | 61.8 |
| 2 | II | 52.0 | 61.9 |

The experimental results are identical.

The catalyst precursor can be handled readily in respect of transport and introduction into the reactor, can easily be converted into the active catalyst and does not form any substances which reduce the catalyst stability and/or the reactivity and/or the selectivity. The cyclooctadiene is hydroformylated to the cyclooctene carbaldehyde during preforming of the catalyst and the second double bond is then hydrogenated, so that cyclooctane carbaldehyde which does not damage the catalyst is formed therefrom after the preformation. (Reference for 1,5-COD: J. Falbe, N. Huppes, Brennstoff-Chemie, 1966, 47, 314-315)

1. A catalyst precursor comprising a rhodium complex represented by formula I 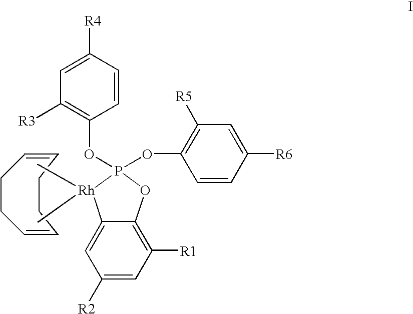
where
R1 to R6=H or a C₁-C₄-alkyl or C₁-C₄-alkoxy radical
or
R1, R2, R4 and R6=H or a C₁-C₄-alkyl or C₁-C₄-alkoxy radical and R3=R5=O, NR where R═H or C₁-C₄-alkyl, or (CH₂)ₙ where n=0-2.

2. The catalyst precursor according to claim 1,
wherein
at least one of the radicals R1 to R6 is a C₁-C₄alkyl radical.

3. The catalyst precursor according to claim 2,
wherein
at least one of the radicals R1 to R6 is a tert-butyl radical.

4. The catalyst precursor according to claim 2,
which
is a complex represented by formula II

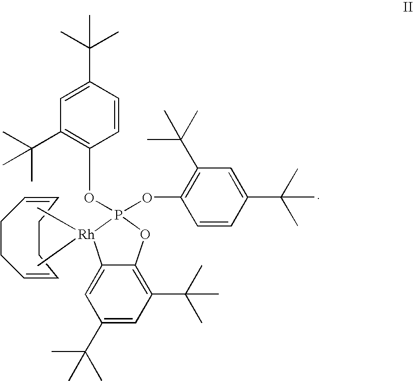

5. The catalyst precursor according to claim 1,
wherein
at least one of the radicals R1 to R6 is a C₁-C₄-alkoxy radical.

6. The catalyst precursor according to claim 4,
wherein
at least one of the radicals R1 to R6 is a methoxy radical.

7. A mixture comprising the catalyst precursor according to claim 1 and at least one organophosphorus ligand.

8. (canceled)

9. A process for the hydroformylation of an olefin, comprising:
reacting said olefin with a gas comprising H₂ and CO in the presence of a catalyst obtained from a catalyst precursor according to claim 1.

10. A process for the hydroformylation of an olefin, comprising:
reacting said olefin with a gas comprising H₂ and CO in the presence of a catalyst obtained from a catalyst precursor according to claim 2.

11. A process for the hydroformylation of an olefin, comprising:
reacting said olefin with a gas comprising H₂ and CO in the presence of a catalyst obtained from a catalyst precursor according to claim 3.

12. A process for the hydroformylation of an olefin, comprising:
reacting said olefin with a gas comprising H₂ and CO in the presence of a catalyst obtained from a catalyst precursor according to claim 4.

13. A process for the hydroformylation of an olefin, comprising:
reacting said olefin with a gas comprising H₂ and CO in the presence of a catalyst obtained from a catalyst precursor according to claim 5.

14. A process for the hydroformylation of an olefin, comprising:
reacting said olefin with a gas comprising H₂ and CO in the presence of a catalyst obtained from a catalyst precursor according to claim 6.

15. A mixture comprising the catalyst precursor according to claim 2 and at least one organophosphorus ligand.

16. A mixture comprising the catalyst precursor according to claim 3 and at least one organophosphorus ligand.

17. A mixture comprising the catalyst precursor according to claim 4 and at least one organophosphorus ligand.

18. A mixture comprising the catalyst precursor according to claim 5 and at least one organophosphorus ligand.

19. A mixture comprising the catalyst precursor according to claim 6 and at least one organophosphorus ligand.

20. The catalyst precursor according to claim 2,
wherein
at least one of radicals R1 to R6 is a C₁-C₄-alkoxy radical.

21. The catalyst precursor according to claim 3,
wherein
at least one of radicals R1 to R6 is a C₁-C₄-alkoxy radical.
